(19) United States
(12) Patent Application Publication
(10) Pub. No.: US 2009/0305375 A1
(43) Pub. Date: Dec. 10, 2009

(54) SUSTAINABLE SUPPLY OF BIOACTIVE MARINE PRODUCTS

(76) Inventors: Russell G. Kerr, Charlottetown (CA); Thomas Brueck, Boca Raton, FL (US)

Correspondence Address: Gregory A. Nelson;Novak Druce & Quigg LLP, 525 Okeechobee Blvd, Suite 1500, West Palm Beach, FL, 33401, US

(21) Appl. No.: 11/917,125

(22) PCT Filed: Jun. 9, 2006
(86) PCT No.: PCT/US06/22462

§ 371 Date: Jun. 9, 2009

Related U.S. Application Data
(60) Provisional application No. 60/689,311, filed on Jun. 10, 2005.
(60) Provisional application No. 60/776,045, filed on Feb. 23, 2006.

(51) Int. Cl. C12P 5/02; C12N 1/20

(52) U.S. Cl. 435/167; 435/252.1

(57) ABSTRACT

The identification and characterization of the sources of terpenes from the soft corals Leptogorgia minimata (LM, formerly Lophogorgia spp., Family: Gorgoniidae), Swifita exertia (SE, Family: Plexauridae), Iciligorgia schrammi (IS, Family: Anthothelidae), Eunicea fusca, Erythropodium caribaeorum or Plexaurella sp. through direct culture of microbial populations derived from coral homogenates.

### FIELD OF THE INVENTION

The invention relates to the identification of bacteria as the biosynthetic origin of bioactive terpenes initially isolated from marine corals, and a cell culture system for producing such compounds.

### BACKGROUND OF THE INVENTION

A number of biologically active compounds with potential commercial applications have been derived from marine organisms. In many cases, the commercial development of these compounds has been hindered because they are often scarce and difficult to obtain. For example, for compounds found in corals, a large amount of coral must be harvested from the environment to obtain amounts necessary for the research and development preceding product introduction as well as for inclusion in products to be sold. Adding to this problem, the structural complexity of many marine organism-derived biologically active compounds (e.g., terpenes), precludes their synthesis by conventional organic chemistry methods.

It is therefore of interest to study microbial association of these deeper water coral species and to ultimately identify the producer of the natural products found in these coral species.

### SUMMARY

The identification and characterization of the sources of terpenes from the soft corals Eunicea fusca, Erythropodium caribaeorum, Pseudopterogorgia elisabethae or Plexaurella sp. through direct culture of microbial populations derived from coral homogenates. The terpene producing microbe are generally identified by cross referencing information from bacterial 16S and fungal 18S ribosomal (r)DNA sequence screens utilizing total DNA extracts from coral, mixed and pure microbial cultures.

In a preferred embodiment, a method of producing terpenes from coral, comprises homogenizing coral sample; isolating bacterial organisms from the coral sample; culturing the isolated bacterial organisms; and, producing terpenes. Preferably, the coral comprises Erythropodium caribaeorum, Eunica fusca, Plexaurella sp. or Pseudopterogorgia elisabethae.

In another preferred embodiment, the bacterial organisms isolated from Erythropodium caribaeorum produce eleutherobin.

In another preferred embodiment, the bacterial organisms isolated from Eunica fusca is Staphylococcus sp and produces fuscol and eunicol.

In another preferred embodiment, the bacterial organisms isolated from Pseudopterogorgia elisabethae produce pseudopterosins.

In yet another preferred embodiment, the isolated bacterial organisms produce terpenes selected from the group consisting of: diterpenes, eleutherobin, erythrolides A and B, desmethyleleutherobin, sarcodyetions A and B, sesquiterpenes—α-curcumene, α-santalene and β-bisabolene; eleutherobin, fuscol, eunicol and pseudopterosins A-Z.

In a preferred embodiment, the isolated bacterial organisms comprise at least one of: Alphaproteobacteria, Betaproteobacteria, Gammaproteobacteria, Deltaproteobacteria, Sphingobacteria, Archaebacteria, Firmicutes, or Staphylococcus sp

In a preferred embodiment, a method of isolating and identifying terpene producers from deep and shallow water corals, comprises: removing contaminants from coral; homogenizing coral; filtering homogenates; culturing filtrates; isolating cultured cells from filtrates; cloning and culturing of individual cells; removing culture supernatants from isolated cells; and, identifying terpenes from clonally cultured cell supernatants. The methods are described in detail in the Examples which follow.

In another preferred embodiment, a method of isolating and identifying terpenes from corals, the method comprises: obtaining and drying corals; extracting compounds from dried coral material; isolating and purifying terpenes from coral extracts; and, identifying terpenes from the deep water corals. The methods are described in detail in the Examples which follow.

In another preferred embodiment, a composition comprises a coral associated bacterium; a Gram negative bacterium; and, lipopolysaccharide (LPS). The lipopolysaccharide can be added in addition to the Gram negative bacteria or the lipopolysaccharide is added instead of the Gram negative bacterium. The ratio of coral associated bacterium:Gram negative bacterium is in the range of about 1:1 up to 1×10⁵:1 as measured by number of cells. The lipopolysaccharide is about 0.1 μg/ml up to about 1 mg/ml.

In a preferred embodiment, the coral associated bacterium comprises at least one of: Alphaproteobacteria, Betaproteobacteria, Gammaproteobacteria, Deltaproteobacteria, Sphingobacteria, Archaebacteria, Firmicutes, or Staphylococcus sp.

In another preferred embodiment, a method of inducing terpene production comprises culturing a composition comprising a coral associated bacterium; a Gram negative bacterium; and/or lipopolysaccharide (LPS); and, incubating said composition from about 24 hrs up to one week; and, isolating the terpenes produced; and, purifying the terpenes. The coral associated bacterium comprises at least one of: Alphaproteobacteria, Betaproteobacteria, Gammaproteobacteria, Deltaproteobacteria, Sphingobacteria, Archaebacteria, Firmicutes, or Staphylococcus sp. Preferably, the ratio of coral associated bacterium:Gram negative bacterium is in the range of 1:1 up to 1×10⁵:1 as measured by number of cells and the lipopolysaccharide is about 0.1 μg/ml up to about 1 mg/ml.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIGS. 1A and 1B are chromatograms showing GC-MS data. FIG. 1A is total ion chromatogram and FIG. 1B is mass spectrum of peak at 14.51 min which is identical to that of an authentic standard of fuscol.

FIGS. 2A and 2B are ¹H-HMR spectra of fuscol. FIG. 2A shows the spectrum of an authentic standard of fuscol; FIG. 2B is a spectrum of fuscol isolated from mixed bacterial culture.

### DETAILED DESCRIPTION

The invention provides for the identification, isolation and characterization of the producer of terpenes from the soft corals Erythropodium caribaeorum, Eunica fusca, Plexaurella sp. or Pseudopterogorgia elisabethae through direct culture of microbial populations derived from coral homogenates. The terpene producing microbe are identified by cross referencing information from bacterial 16S and fungal 18S rDNA sequence screens utilizing total DNA extracts from coral, mixed and pure microbial cultures.

### DEFINITIONS

As utilized in accordance with the present disclosure, the following terms, unless otherwise indicated, shall be understood to have the following meanings:

The term “terpene” as used herein refers to all terpene compounds including precursors and derivatives thereof. For example, diterpenes, eleutherobin, erythrolides A and B, desmethyleleutherobin, sarcodyctions A and B, sesquiterpenes—α-curcumene, α-santalene and β-bisabolene; eleutherobin, fuscol, eunicol and pseudopterosins A-Z.

“Patient” refers to a mammal, which is preferably human.

“Pharmaceutically acceptable salt” refers to a salt of a compound that is pharmaceutically acceptable and that possesses the desired pharmacological activity of the parent compound.

“Pharmaceutically acceptable carrier” refers to a diluent, adjuvant, excipient or vehicle with which a compound is administered.

“Pharmaceutical composition” as used herein refers to at least one terpene and a pharmaceutically acceptable carrier with which the terpene is administered to a patient.

“Therapeutically effective amount” means the amount of a compound that, when administered to a patient for controlling a condition, such as for example, inflammation, pain, pro- and anti-angiogenesis and the like, is sufficient to effect such control.

The “therapeutically effective amount” will vary depending on the compound, the severity of the condition causing the need to control angiogenesis and the age, weight, etc., of the patient to be treated.

By the phrase “a sample enriched for bacteria isolatable from a coral” is meant that the sample has a higher ratio of bacteria to non-bacterial organisms than does an unfractionated homogenate of the coral.

### Microbial Sources of Bioactive Natural Products

Studies on marine microorganisms face various problems. First, the taxonomy of marine bacteria and fingi is very poorly defined so that taxonomic identifications are difficult to confirm. Second, the requirements for microorganisms to be considered marine species are poorly understood and defined. Marine bacteria often need seawater to grow whereas most marine fingi have no requirements at all. Also, some species may have been isolated from a marine environment but are taxonomically identical to terrestrial species. Third, most marine microorganisms are difficult to grow and it might be assumed that those are the strict marine ones and thus the ones that might produces original new metabolites. However, special media have been developed that aim to overcome this problem. (See, the Examples which follow). Additionally, metabolic changes might occur in cultured marine species that lead to quantitatively and/or qualitatively modified natural products due to unsatisfied micronutrient requirements in the culture medium. Finally, closely related chemicals my be produced by only distantly related microorganisms.

So far, studies concerning bacteria and fungi from marine invertebrates and other marine sources have approached the preceding problems with two different strategies. A random approach uses any available marine material for microbial isolation while the second utilizes specified marine targets with defined purposes in order to study the chemistry involved in the host/microorganisms associations. While monoterpenes, sesterterpenes, triterpenes, steroid and triterpene saponines and polyoxygenated steroids have not yet been found in marine microbes, diterpenes, sesquiterpenes and carotenes have been described. Among those, okadaxanthine has been isolated from Pseudomonas sp., strain KK 10206C obtained from a homogenate from Halichondria okadai. Gorgonians, also known as sea whips, sea fans or sea plumes, are prominent members of tropical and subtropical habitats world wide. In the Bahamas and Florida, gorgonians represent an estimated 38% of the known fauna with over 195 species documented from the families Briareidae, Plexauridae and Gorgoniidae. These organisms have proven to be a prolific source of novel bioactive natural products, particularly terpenes, which exhibit a range of biomedical activities (Fenical 1987, Rodriguez 1995). Two such examples include fuscol/fuscosides, and eleutherobin.

In another preferred embodiment, the invention provides for isolation, characterization and culturing of symbiotic bacteria and fungi associated with terpene production in deep water soft coral. Preferably, the terpene biosynthetic capability of mixed microbial broth cultures is evaluated by determining bacterial and fungal species contained in mixed cultures by 16S and 18S rDNA sequence screens.

In another preferred embodiment, the terpene biosynthetic capability of pure microbial cultures derived from solid agar plates is evaluated and pure bacterial and fungal cultures are characterized using 16S and 18S rDNA sequence screens respectively.

In another preferred embodiment, all culturable and “uncultivable” microbes associated with deep water coral are characterized using total coral DNA extracts in combination with bacterial 16S and fungal specific 18S rDNA sequence screening.

In another preferred embodiment, terpene producers are isolated and characterized by cross referencing information of 16S and 18S rDNA sequence screens obtained for microbes isolated with biosynthetic data generated for pure and mixed microbial cultures.

In another preferred embodiment, the identified terpene metabolites are isolated and characterized.

In another preferred embodiment, the identification and culturing of terpene producing microbes are developed as sustainable bioactive terpene producers. Without wishing to be bound by theory, terpenes are produced by symbiotic microorganisms found in soft corals in the absence of dinoflagellates, that these organisms are culturable and that pure microbial cultures are able to produce terpenes over prolonged time periods.

The identification and characterization of the sources of terpenes from soft corals Eunicea fusca, Erythropodium caribaeorum, Pseudopterogorgia elisabethae or Plexaurella sp. through direct culture of microbial populations derived from coral homogenates. The terpene producing microbes are generally identified by cross referencing information from bacterial 16S and fungal 18S ribosomal (r)DNA sequence screens utilizing total DNA extracts from coral, mixed and pure microbial cultures.

In a preferred embodiment, a method of producing terpenes from coral, comprises homogenizing coral sample; isolating bacterial organisms from the coral sample; culturing the isolated bacterial organisms; and, producing terpenes. Preferably, the coral comprises Erythropodium caribaeorum, Eunica fusca, Plexaurella sp. or Pseudopterogorgia elisabethae.

In another preferred embodiment, the bacterial organisms isolated from Erythropodium caribaeorum produce eleutherobin.

In another preferred embodiment, the bacterial organisms isolated from Eunica fusca is Staphylococcus sp and produces fuscol and eunicol.

In another preferred embodiment, the bacterial organisms isolated from Pseudopterogorgia elisabethae produce pseudopterosins.

In yet another preferred embodiment, the isolated bacterial organisms produce terpenes selected from the group consisting of: diterpenes, eleutherobin, erythrolides A and B, desmethyleleutherobin, sarcodyctions A and B, sesquiterpenes—α-curcumene, α-santalene and β-bisabolene; eleutherobin, fuscol, eunicol and pseudopterosins A-Z.

In a preferred embodiment, the coral associated bacteria or bacteria isolated from coral the isolated bacterial organisms comprise at least one of: Alphaproteobacteria, Betaproteobacteria, Gammaproteobacteria, Deltaproteobacteria, Sphingobacteria, Archaebacteria, Firmicutes, or Staphylococcus sp.

In a preferred embodiment, a method of isolating and identifying terpene producers from deep and shallow water corals, comprises: removing contaminants from coral; homogenizing coral; filtering homogenates; culturing filtrates; isolating cultured cells from filtrates; cloning and culturing of individual cells; removing culture supernatants from isolated cells; and, identifying terpenes from clonally cultured cell supernatants. The methods are described in detail in the Examples which follow.

In another preferred embodiment, a method of isolating and identifying terpenes from corals, the method comprises: obtaining and drying corals; extracting compounds from dried coral material; isolating and purifying terpenes from coral extracts; and, identifying terpenes from the deep water corals. The methods are described in detail in the Examples which follow.

In another preferred embodiment, a composition comprises a coral associated bacterium; a Gram negative bacterium; and, lipopolysaccharide (LPS). The lipopolysaccharide can be added in addition to the Gram negative bacteria or the lipopolysaccharide is added instead of the Gram negative bacteria. The ratio of coral associated bacterium:Gram negative bacterium is in the range of about 1:1 up to 1×10⁵:1 as measured by number of cells. The lipopolysaccharide is about 0.1 μg/ml up to about 1 mg/ml.

In a preferred embodiment, the coral associated bacterium comprises at least one of: Alphaproteobacteria, Betaproteobacteria, Gammaproteobacteria, Deltaproteobacteria, Sphingobacteria, Archaebacteria, Firmicutes, or Staphylococcus sp.

In another preferred embodiment, a method of inducing terpene production comprises culturing a composition comprising a coral associated bacterium; a Gram negative bacterium; and/or lipopolysaccharide (LPS); and, incubating said composition from about 24 hrs up to one week; and, isolating the terpenes produced; and, purifying the terpenes. The coral associated bacterium comprises at least one of: Alphaproteobacteria, Betaproteobacteria, Gammaproteobacteria, Deltaproteobacteria, Sphingobacteria, Archaebacteria, Firmicutes, or Staphylococcus sp. Preferably, the ratio of coral associated bacterium:Gram negative bacterium is in the range of 1:1 up to 1×10⁵:1 as measured by number of cells and the lipopolysaccharide is about 0.1 μg/ml up to about 1 mg/ml.

The compounds in accordance with the present invention are useful in the treatment of rheumatoid arthritis, osteoarthritis, rheumatic carditis, collagen and auto-immune diseases such as myasthenia gravis, allergic diseases, bronchial asthma and ocular and skin inflammatory diseases such as poison ivy. The compounds are also useful in treating proliferative diseases such as psoriasis.

The compounds are potent non-narcotic analgesics and may be used to alleviate pain resulting from traumatic injury or acute progressive disease, such as post-operative pain, burns, or other conditions involving a coincident inflammation.

In one preferred embodiment, the compounds are used as anesthetics.

### Effective Amounts

A compound of the present invention may be administered in a therapeutically effective amount to a mammal such as a human. A therapeutically effective amount may be readily determined by standard methods known in the art. As defined herein, a therapeutically effective amount of a compound of the invention ranges from about 0.1 to about 25.0 mg/kg body weight, preferably about 1.0 to about 20.0 mg/kg body weight, and more preferably about 10.0 to about 20.0 mg/kg body weight. Preferred topical concentrations include about 0.1% to about 20.0% in a formulated salve. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of the compound can include a single treatment or, preferably, can include a series of treatments.

In a preferred example, a subject is treated with a compound of the invention in the range of between about 0.1 to about 25.0 mg/kg body weight, at least one time per week for between about 5 to about 8 weeks, and preferably between about 1 to about 2 weeks. It will also be appreciated that the effective dosage of the compound used for treatment may increase or decrease over the course of a particular treatment.

Changes in dosage may result and become apparent by standard diagnostic assays known in the art. In some conditions chronic administration may be required.

The pharmaceutical compositions of the invention may be prepared in a unit-dosage form appropriate for the desired mode of administration. The compositions of the present invention may be administered for therapy by any suitable route including oral, rectal, nasal, topical (including buccal and sublingual), vaginal and parenteral (including subcutaneous, intramuscular, intravenous and intradermal). It will be appreciated that the preferred route will vary with the condition and age of the recipient, the nature of the condition to be treated, and the chosen active compound.

It will be appreciated that the actual dosages of the agents used in the compositions of this invention will vary according to the particular complex being used, the particular composition formulated, the mode of administration, and the particular site, host, and disease being treated. Optimal dosages for a given set of conditions may be ascertained by those skilled in the art using conventional dosage-determination tests in view of the experimental data for a given compound. Administration of prodrugs may be dosed at weight levels that are chemically equivalent to the weight levels of the fully active forms.

The compounds of the invention can be incorporated into pharmaceutical compositions suitable for administration. Pharmaceutical compositions of this invention comprise an therapeutically effective amount of any one or more compounds produced and isolated from coral-associated bacteria and an inert, pharmaceutically acceptable carrier or diluent. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Supplementary active compounds include other pseudopterosins and seco-pseudopterosins such as those described in U.S. Pat. Nos. 4,745,104, 4,849,410, and 5,624,911, all of which are herein incorporated by reference. Supplementary compounds also include hydrocortisone, cox inhibitors such as indomethacin or salicylates, fixed anesthetics such as lidocaine, opiates, and morphine.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

### Materials and Methods

Collection of Coral

Coral samples of interest were collected by SCUBA from locations in Florida and the Bahamas. Specifically, Pseudopterogorgia elisabethae (PE) and Plexaurella sp. (PS) were collected at Sweetings Cay at a depth of 30 ft in the Bahamas, and Eunicea fusca (EF) and Erythropodium caribaeorum (EC) were collected at a depth of 30 ft off Pompano Beach, Fla. All corals were kept alive in aerated water containers until further processing in the lab (no longer than 3 h after collection). All collections were conducted by certified ‘scientific divers’ in compliance with AAUS (American Academy of Underwater Sciences) regulations and all collections were carried out with the appropriate collection permits from the State of Florida or the Government of The Bahamas.

### Example 1

### Confirmation of the Presence of Terpenes in Corals: PE, PS, EF and EC

The major terpenes of the corals PE, PS, EF and EC were purified from the corals by standard protocols according to literature procedures (J. Org. Chem. 51, 5140, 1986; J. Am. Chem. Soc. 119, 8744, 1997; J. Org. Chem. 56, 3153, 1991). The terpenes were isolated to provide authentic standards to aid in the identification of these compounds in microbial cultures from their respective corals. Table 1 below identifies the major terpenes from each coral.

TABLE 1

| | |
|---|---|
| PE | pseudopterosins A, B, C & D or G, H, I & J |
| | (identities vary with collection site |
| PS | α-curcumene, β-bisabolene |
| EF | fuscol, eunicol |
| EC | eleutherobin, erythrolide A |

### Example 2

### Evidence for a Bacterial Origin of Terpenes in Corals PE, PS, EF and EC

The general approach to determine if a bacterium from the corals was capable of terpene biosynthesis was to generate a mixed bacterial preparation from each coral and conduct a biochemical assay to assess terpene biosynthetic capability and/or examine the terpene content. In each case, the gorgonian tissue (typically 50 g) was homogenized in 100 mL filtered sea water (FSW), filtered through cheese cloth and repeatedly centrifuged at low speed (1,000 g). The supernatant was then filtered to remove eukaryotic cells (gorgonian and dinoflagellate) by pre-filtration through 5 μM, and then through 1.2 μM and 0.8 μM. Filtrates from the 0.8 μM filtrations were either: (a) pelleted by centrifugation at 10,000-35,000 g; the pellet is then suspended in filtered sea water (FSW), media or buffer for biosynthetic examination or the inoculation of cultures; (b) further filtered through 0.45 μM and 0.2 μM. In such cases, the bacteria were washed off the filter by gentle sonication in FSW, buffer or culture media.

Microbial preparations from the corals listed above were mostly composed of Gammaproteobacteria and Alphaproteobacteria, with Sphingobacteria and Archaebacteria present in lesser amounts. PCR of DNA preparations from the microbial preparations, and Symbiodinium (isolated by known methods) were performed using coral-, bacteria-, and Symbiodinium-specific primers (coral ITS region, bacteria 16S rDNA region, Symbiodinium ITS region). With coral and Symbiodinium primers the only bands observed were in the Symbiodinium DNA fraction, however, using bacteria-specific primers amplicons were observed for all three fractions tested.

Biosynthetic data: A microbial preparation of the gorgonian corals PE, PS, EF and EC was suspended in 40 mL FSW and incubated with 2 μCi ³H-geranylgeranyl diphosphate ([C₁—³H] GGPP) for 24 hours. The terpenes were extracted from the incubation mixture and demonstrated to be radioactive by scintillation counting. The DNA analysis described above confirms that the microbial preparations were devoid of gorgonian and dinoflagellate cells, thus indicating that the observed transformation of labeled GGPP is due to the action of bacteria. (A Bradford assay of the washes of the bacterial preparation confirmed that there is no protein present.) The radioactivity incorporation data for each gorgonian are shown below in Table 2.

TABLE 2
Incorporation of radioactivity from incubation of 2 μCi
[C₁—³H] GGPP with microbial preparations of: PE, PS,
EF, EC, LM, SE and IS to the major terpenes.

| | | | |
|---|---|---|---|
| Pseudopterogorgia elisabethae | pseudopterosin G | 5,440 | DPM |
| | pseudopterosin H | 1,800 | DPM |
| | pseudopterosin I | 3,000 | DPM |
| | pseudopterosin J | 3,860 | DPM |
| Eunicea fusca | fuscol | 180 | DPM |
| | eunicol | 2,290 | DPM |
| | eunicene A | 1,980 | DPM |
| | fuscoside A | 5,650 | DPM |
| Erythropodium caribaeorum | erythrolide A | 940 | DPM |
| | eleutherobin | 520 | DPM |
| Plexaurella sp. | α-curcumene | 4,000 | DPM |
| | β-bisabolene | 6,800 | DPM |

### Example 3

### Demonstration of Terpene Production in Mixed Bacterial Cultures

General culture conditions: Prior to inoculation, the coral samples were washed 3 times in sterile sea water to remove any contaminants present in the sea water and on the surface of the coral before further processing. Samples (300 g wet weight) were homogenized under sterile conditions (in sterile hood) in phosphate buffered saline (PBS, 150 mL) and filtered through cheese cloth to remove coral skeletal particulates before inoculation. The filtrate was used to inoculate a series of culture media, namely: YM, Nutrient and Marine broth (Fisher Scientific: Difco Nutrient Broth-DF0003-17-8, Difco Marine Broth-DF0791-17-4, Difco YM Broth-DF0711-17-1).

Initially, a heavy inoculum of the coral extract was used, which contains several humoral factors (compounds) derived from the coral host. The presence of humoral factors in broth cultures may host a variety of culturable bacterial and fungal microbes not achieved with conventional plate culture. For direct culturing of associated coral microbes 10 ml broth cultures with 1 ml coral homogenate were inoculated and grown at 25° C. and 250 rpm for 6 days. A gradual scale up was performed after 6 days to 500 ml. During the gradual scale up procedure coral specific humoral factors are slowly diluted out to enable the microbial communities supported by each medium to adjust to the absence of the host.

To test the mixed microbial cultures for the presence of signature metabolites (i.e. terpenes isolated from the host coral), aliquots of the broth were extracted and purified using procedures described infra. The presence or absence of a particular signature terpene was assessed by comparing equivalent HPLC profiles and NMR spectra of the broth with that of authentic standards. The biosynthetic capability of each mixed culture was determined by quantitatively measuring target terpene production over time using previously established HPLC methods. Quantitative terpene production is then correlated with microbial growth curves, determined by using broth absorbance at 600 nm. Following identification of a mixed culture that produces the terpenes found in the coral extracts, further characterization of the microbial population in the mixed broth culture is conducted using subcloning, PCR/RFLP in combination with bacterial 16S and fungal 18S rDNA sequence analysis. These data are correlated with those found in the solid agar plates and microbial sequences deduced from direct total DNA isolates of each coral species. Correlation of the data is used to identify the producer of the terpenes under investigation.

### Example 4

### Demonstration of Terpene Biosynthetic Capabilities of a Mixed Microbial Culture from PE—Evidence for a Bacterial Source of Pseudopterosins

Microbial preparations: The PE tissue (typically 50 g) was homogenized in 100 mL filtered sea water (FSW), filtered through cheese cloth and centrifuged ca. 10 times at low speed (1,000×g). The supernatant from was filtered to remove eukaryotic cells (gorgonian and dinoflagellate) by pre-filtration through 20 and 5 μM, and then, filtration through 1.2 μM and/or 0.8 μM. The first pellet consists of Symbiodinium cells which were purified through a Percoll (Amersham Biosciences, cat. no. 17-0891-01) step gradient of 30 and 70% Percoll, followed by several washes with FSW by centrifugation; this constitutes the Symbiodinium fraction (Chem. & Biol. 10, 1051, 2003). Filtrates from either the 5 μM, 1.2 μM or 0.8 μM filtrations can then be pelleted by centrifugation at forces of 10,000-35,000×g; this constitutes the microbial fraction (minus Symbiodinium). The bacterial pellet was then suspended in FSW, media or buffer for biosynthetic examination or the inoculation of cultures.

Production of pseudopterosins in microbial cultures from P. elisabethae: Mixed cultures of the bacterial community from P. elisabethae were established under the following conditions. In a sterile 500 ml Erlenmeyer flask Difco Nutrient Broth (Cat. No. 003-17-8) was prepared in an appropriate concentration (8 grams/liter) or Difco Marine Broth (Beckton Dickinson Cat. No. 297-11-0) in a concentration of 37.4 grams/liter using a Bugstopper™ as the container closure device (Beckton Dickinson Cat. No. 3713-3010), and autoclaving at 121° C. for 30 minutes/liter. After the broth cooled, it was inoculated in a sterile environment using sterile techniques with ca. 20 mg of bacterial or Symbiodinium pellet. Cultures were maintained at 27.5° C. (or 25-30° C.) in static conditions. Four cultures were started (Table 3).

TABLE 3

| Name | Media | Microbial Source |
|---|---|---|
| PE8 | Nutrient Broth (NB) | Symbiodinium sp. |
| PE9 | Marine Broth (MB) | Symbiodinium sp. |
| PE10 | Nutrient Broth (NB) | 5 μm 16000 × g Pellet |
| | | No zoo (confirmed by PCR) |
| PE11 | Marine Broth (MB) | 5 μm 16000 × g Pellet |
| | | No zoo (confirmed by PCR) |

Extraction and Examination of Bacterial Cultures: 25-30 mL of culture suspension was treated with 5 mL of NaCl and 25-30 mL of methanol followed by 1:1 methanol:water and was then partitioned with methylene chloride. The methylene chloride layer was rotovaped and the residue re-suspended in 75:25 hexanes ethyl acetate for analysis by normal phase HPLC. The amount of pseudopterosins present was determined by integration of peak areas. Cultures were initially analyzed by HPLC for the presence of pseudopterosin G, and samples that showed peaks with the same retention time and UV spectra as pseudopterosin G were further analyzed by normal phase APCI-LC/MS.

Results of chemical analysis of cultures: Examination of cultures over time confirmed the production of pseudopterosin G. For cultures PE8, PE10, and PE11 at one month, pseudopterosin G was confirmed by HPLC (and LC/MS) at a concentration of 0.9, 0.37 and 0.22 mg/L, respectively. Quantification was determined by comparison of HPLC peak areas with a standard curve from authentic samples. HPLC chromatograms and diode-array HPLC analysis of culture extracts confirm the presence of pseudopterosin G. APCI-LC/MS of cultures PE8, PE10, and PE 11 were shown to contain pseudopterosin G with m/z of 445.

Biosynthetic Capability of PE8 Culture: A 4.5 month-old culture (PE8) was examined for pseudopterosin biosynthetic capability by incubating with 2 μCi ³H-GGPP for 72 hours. The incubation mixture was extracted (as above) and the methylene chloride layer analyzed by normal phase HPLC. Pseudopterosin G collected from HPLC was re-injected and 5 fractions collected (2 fractions before pseudopterosin G peak, pseudopterosin G and 2 fractions after pseudopterosin G peak. The pseudopterosin G peak collected was radioactive following scintillation counting (1,720 DPM).

Microbial analysis of the cultures: The composition of the bacterial population present in PE8, PE10, and PE11 was analyzed by 16S rDNA analysis. This DNA was PCR-amplified, cloned, digested by the restriction enzyme HhaI, subjected to restriction fragment length polymorphism (RFLP) analysis, and sequenced. Also the presence of contaminating coral, fungi, and Symbiodinium DNA was analyzed by PCR. Molecular analysis of the cultures showed the presence of bacteria in all three cultures and an almost negligible fungal presence in PE10. No coral or Symbiodinium DNA was present in the cultures. Phylogenetic analysis showed a concentration of four types of Proteobacteria (alpha, beta, gamma, delta) and a single member of the Firmicutes group (Table 4). A common group for all three cultures was the Gamma-Proteobacteria.

TABLE 4

| Culture | Strain | Group | ID |
|---|---|---|---|
| PE8 | PE-01A | Gammaproteobacteria | Stenotrophomonas sp. |
| | PE-01B | Betaproteobacteria | Delftia sp. |
| | PE-01C | Gammaproteobacteria | Pseudomonas sp. |
| | PE-01D | Betaproteobacteria | Delftia sp. |
| | PE-01E | Betaproteobacteria | Delftia sp. |
| | PE-01F | Gammaproteobacteria | Stenotrophomonas sp. |
| | PE-01G | Gammaproteobacteria | Stenotrophomonas sp. |
| PE10 | PE-01S | Gammaproteobacteria | Acinetobacter sp. |
| | PE-01T | Gammaproteobacteria | Acinetobacter sp. |
| | PE-01U | Gammaproteobacteria | Acinetobacter sp. |
| | PE-01V | Gammaproteobacteria | Acinetobacter sp. |
| | PE-01W | Gammaproteobacteria | Acinetobacter sp. |
| | PE-01Y | Gammaproteobacteria | Acinetobacter sp. |
| | PE-01Z | Firmicutes | Bacillus sp. |
| PE11 | PE-01AA | Deltaproteobacteria | Desulfovibrio sp. |
| | PE-01BB | Deltaproteobacteria | Desulfovibrio sp. |
| | PE-01CC | Deltaproteobacteria | Desulfovibrio sp. |
| | PE-01DD | Gammaproteobacteria | Marinobacter sp. |
| | PE-01EE | Alphaproteobacteria | Roseobacter sp. |
| | PE-01FF | Deltaproteobacteria | Desulfovibrio sp. |
| | PE-01GG | Deltaproteobacteria | Desulfovibrio sp. |
| | PE-01HH | Alphaproteobacteria | Roseobacter sp. |
| | PE-01II | Alphaproteobacteria | Roseobacter sp. |
| | PE-01JJ | Gammaproteobacteria | Marinobacter sp. |
| | PE-01KK | Gammaproteobacteria | Marinobacter sp. |
| | PE-01LL | Alphaproteobacteria | Roseobacter sp. |

### Example 5

### Demonstration of Terpene Biosynthetic Capabilities of a Mixed Microbial Culture from EF—Evidence for a Bacterial Source of Fuscol, Eunicol and Fuscosides

Mixed cultures of the microbial community from E. fusca were established under the following conditions. Freshly collected coral was macerated in FSW, and filtered through 3 layers of cheesecloth twice to remove large particulate matter, and the filtrate was centrifuged at 2,000×g for 4 minutes to pellet coral cells and zooxanthellae. The supernatant was decanted, and sterile filtered through an 8.0 μm pre-filter with a vacuum filtration device. The filtrate was sterile filtered through a 5.0 μm and subsequently filtered through a 0.45 micron filter and the retentate retained.

Media Preparation and Inoculations: In a sterile 500 ml Erlenmeyer flask Difco Nutrient Broth (Cat. No. 003-17-8) was prepared in an appropriate concentration (8 grams/liter) or Difco Marine Broth (Beckton Dickinson Cat. No. 297-11-0) in a concentration of 37.4 grams/liter using a Bugstopper™ as the container closure device (Beckton Dickinson Cat. No. 3713-3010), and autoclaving at 121° C. for 30 minutes/liter. After the broth cooled, it was inoculated in a sterile environment using sterile techniques with ca. 20 mg of bacterial or Symbiodinium pellet. Cultures were maintained at 27.5° C. (or 25-30° C.) in static or shaking conditions.

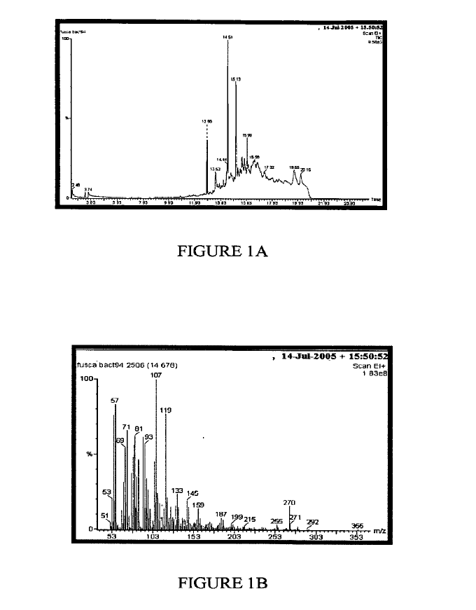

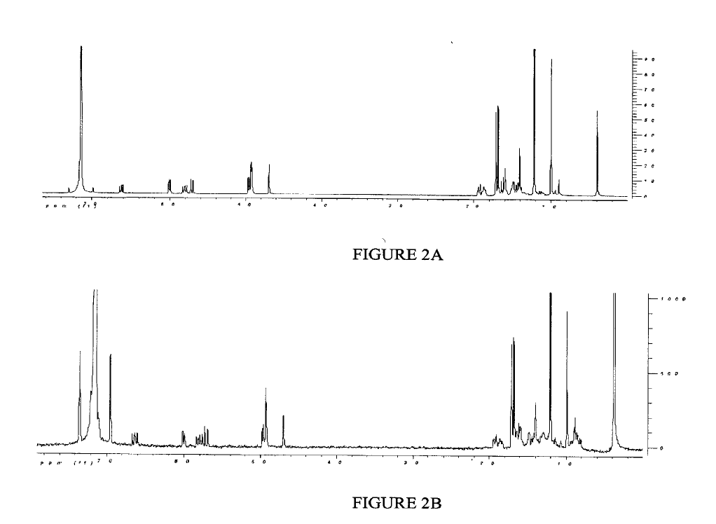

Extraction and Examination of Bacterial Cultures: A 20-25 ml aliquot of the culture was lyophilized to dryness, extracted with 9:1 methanol:water and partitioned with hexanes. The hexanes layer was rotovaped and the residue re-suspended in methanol for analysis by HPLC. The amounts of fuscol and eunicol were determined by integration of HPLC peak areas. Examination of cultures over time confirmed the production of fuscol and eunicol. Fuscol and eunicol was readily detected by HPLC at 2 months, at a combined concentration of 2.0 mg/L, and after 5 months, the presence of fuscol and eunicol was confirmed by HPLC (and GC/MS) at a combined concentration of 18.0 mg/L. Quantification was determined by comparison of HPLC peak areas with a standard curve from authentic samples. The identity of fuscol and eunicol was confirmed by GC-MS analysis (FIG. 1) and ¹H-HMR (FIG. 2) provided further confirmation of the presence of fuscol.

DNA from the cultures was PCR-amplified using 16S rDNA bacteria-specific primers. The PCR amplicons were cloned, restriction digested, and sequenced to determine their bacterial identity. From RFLP analysis and from sequencing it was determined that there were several types of bacteria growing in these cultures and that ca. 80% were members of the Gammaproteobacteria and Alphaproteobacteria. Members of the Sphingobacteria and Archaebacteria were also observed but in lower concentrations.

### Example 6

### Isolation of the Bacterium Responsible for Terpene Biosynthesis from the Mixed Bacterial

The fuscol-producing mixed culture was streaked onto cell culture plates, colonies were picked and used to inoculate liquid cultures in Marine Broth. A total of ˜600 pure cultures were screened for the presence of fuscol/eunicol by GC-MS and HPLC. One such culture was identified and is characterized as described below. Microscopic examination of the fuscol-producing bacterium indicated that this was a pure culture by visual examination which was a Gram (+) cocci. To identify the cocci bacterium, DNA was obtained from pure liquid culture and amplified with 16S primers, cloned and sequenced.

Liquid culture was streaked onto plates, individual colonies PCRd, cloned and sequenced. All sequences had 99.5% similarity to a Gram (+) bacterium—identified as a Staphylococcus sp (EF-S-01). There are no reports of terpene production from a Staphylococcus and thus this finding is highly novel and significant.

Sequence Information:

R-NCN1 sequence exported from R-NCN1_A11.ab1

(SEQ ID NO: 1)

TTTTCCgtCGAGCGAACAGAAGAGGAGCTTGCTCCTCTGACGTTAGCGGC
GGACGGGTGAGTAACACGTGGATAACCTACCTATAAGACTGGGATAACTT
CGGGAAACCGGAGCTAATACCGGATAATATATTGAACCGCATGGTTCAAT
AGTGAAAGACGGTTTTGCTGTCACTTATAGATGGATCCGCGCCGCATTAG
CTagTTggTAAGGTAACGGCTTACCAAGGCAACGATGCGTAGCCGACCTG
AGAGGGTGATCGGCCACACTGGAACTGAGACACGGTCCAGACTCCTACGG
GAGGCAGCAGTAGGGAATCTTCCGCAATGGGCGAAAGCCTGACGGAGCAA
CGCCGCGTGAGTGATGAAGGTCTTCGGATCGTAAAACTCTGTTATTAGGG
AAGAACAAATGTGTAAGTAACTATGCACGTCTTGACGGTACCTAATCAgA
AAGCCACGGCTAACTACGTGCCAgCAGCCGCGGTATACgTAGTGGCAAGC
GTTATCCGGAATTATTGGGCGTAAAGCGCGCGTAGGCGGTTTTTTAAgTC
TGATGTGAAAgCCaCGGCTCACCGTGGAGGGTCATTGGAACTGGAAA

### Example 6

### Optimization of Growth Conditions for the Diterpene-Producing Bacterium, Staphylococcus sp. EF-S-01 Isolated from Eunicea Fusca:

The growth of the Staphylococcus sp. isolated from Eunicea fusca (EF-S-01) was optimized by varying conditions described below.

Nutrient-rich medium:
Marine agar medium with different initial pHs, 4, 5, 6, 7, 8 and 9
Marine agar using filtered sea water with different pHs range (pH 4, 5, 6, 7, 8 and 9)
Marine agar using distilled water with different pHs range (pH 4, 5, 6, 7, 8 and 9)
Nutrient-poor medium:
Marine agar, diluted 1:10, using sea water with different initial pHs
Marine agar, diluted 1:10, using distilled with different initial pHs

EF-S-01 was streaked on the above different media and incubated at 25° C., 30° C. and 37° C., and the growth was monitored every day for appearance of colonies. EF-S-01 showed the best growth after 24 h incubation at 37° C. in diluted (1:10) Marine agar medium in sea water with a pH of 5.5.

### Example 7

### Induction of Diterpene Production by Staphylococcus Sp. EF-S-01

Biodiversity studies of coral associated-bacteria, indicated that Gram negative bacteria constitute high percentage of the coral associated-bacteria. Assuming that the terpenes from E. fusca (fuscol, eunicol and fuscosides) are produced as antibiotics active against a variety of Gram negative bacteria, experiments were conducted to upregulate terpene production by treating cultures of EF-S-01 with a Gram negative bacterium or LPS which mimics the presence of a Gram negative bacterium. The Staphylococcus strain was inoculated in 50 ml medium, incubated overnight at 37° C. at 150 r.p.m and then transferred to 250 ml medium and incubated at the same conditions. A 5 ml aliquot of a culture of Pseudomonas aeruginosa grown for 12 hrs was added to the above Staphylococcus culture and incubated for a further 72 hr. Relative to a control of a pure culture of EF-S-01, the treated culture had significantly higher concentrations of terpenes as determined by GC-MS. Control—0.8 mg/L fuscol & eunicol; treated sample—2.5 mg/L.

Induction of terpene production with addition of lipopolysaccharides (LPS) extracted from Gram negative bacteria: LPS is the major structural difference between the cell membrane of Gram negative and Gram positive bacteria. LPS (extracted from E. coli) was added (20 μg/ml) to a culture of Staphylococcus sp. (EF-S-01) and the incubation was continued for 72 h. Relative to a control of a pure culture of EF-S-01, the treated culture had significantly higher concentrations of terpenes as determined by GC-MS. Control—0.9 mg/L fuscol & eunicol; treated sample—2.0 mg/L.

### Other Embodiments

This description has been by way of example of how the compositions and methods of the invention can be made and carried out. Various details may be modified in arriving at the other detailed embodiments, and many of these embodiments will come within the scope of the invention. Therefore, to apprise the public of the scope of the invention and the embodiments covered by the invention, the following claims are made.

What is claimed is:

1. A method for producing a terpene, the method comprising:
providing a sample enriched in bacteria isolatable from a coral and capable of producing terpenes; and,
isolating the terpene from the bacterial sample.

2. The method of claim 1, wherein the coral comprises Erythropodium caribaeorum, Eunica fusca, Plexaurella sp. or Pseudopterogorgia elisabethae.

3. The method of claim 1, wherein the isolated bacterial organisms comprise at least one of: Alphaproteobacteria, Betaproteobacteria, Gammaproteobacteria, Deltaproteobacteria, Sphingobacteria, Archaebacteria, Firmicutes, or Staphylococcus sp.

4. The method of claim 1 wherein the bacterial sample is cultured in an appropriate medium facilitating growth and production of terpenes.

5. The method of claim 2 wherein the bacteria are isolated from Eunicea fusca and the terpene is selected from the group consisting of: fuscol, eunicol, fuscosides A and B.

6. The method of claim 2 wherein the bacteria are isolated from Pseudopterogorgia elisabethae and the terpene is selected from the group consisting of: pseudopterosins A-Z.

7. The method of claim 2 wherein the bacteria are isolated from Erythropodium caribaeorum and the terpene is selected from the group consisting of: eleutherobin, desmethyleleutherobin, sarcodyctions A and B, erythrolides A and B.

8. The method of claim 2 wherein terpene biosynthesis is induced through the addition of lipopolysaccharide from Gram negative bacteria.

9. The method of claim 1, wherein the isolated bacterial organisms produce terpenes selected from the group consisting of: diterpenes, eleutherobin, erythrolides A and B, desmethyleleutherobin, sarcodyctions A and B, sesquiterpenes—α-curcumene, α-santalene and β-bisabolene; eleutherobin, fuscol, eunicol and pseudopterosins A-Z.

10. A composition comprising:
a coral associated bacterium;
a Gram negative bacterium; and,
lipopolysaccharide (LPS).

11. The composition of claim 10, wherein the lipopolysaccharide is added in addition to the Gram negative bacteria.

12. The composition of claim 10, wherein the lipopolysaccharide is added instead of the Gram negative bacteria.

13. The composition of claim 10, wherein the coral associated bacterium comprises at least one of: Alphaproteobacteria, Betaproteobacteria, Gammaproteobacteria, Deltaproteobacteria, Sphingobacteria, Archaebacteria, Firmicutes, or Staphylococcus sp.

14. A method of inducing terpene production comprising:
culturing a composition comprising a coral associated bacterium; a Gram negative bacterium; and/or lipopolysaccharide (LPS); and,
incubating said composition from about 24 hrs up to one week; and,
isolating the terpenes produced; and,
purifying the terpenes.


SEQUENCE LISTING (1 sequence)

SEQ ID NO: 1 - DNA, 597 bp, Staphylococcus sp. EF-S-01
ttttccgtcgagcgaacagaagaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtatacgtagtggcaagcgttatccggaattattgggcgtaaagcgcgcgtaggcggttttttaagtctgatgtgaaagccacggctcaccgtggagggtcattggaactggaaa